(19) United States
(12) Patent Application Publication
(10) Pub. No.: US 2024/0245855 A1
(43) Pub. Date: Jul. 25, 2024

(54) MEDICATION BLADDER FOR MEDICATION STORAGE

(71) Applicant: Willow Laboratories, Inc., Irvine, CA (US)

(72) Inventors: Hung The Vo, Fountain Valley, CA (US); Sai Kong Frank Lee, Irvine, CA (US); Tuan Minh Tran, Irvine, CA (US); Amir K. Dabiri-Asgari, Irvine, CA (US); Hossein Derakhshandeh, Irvine, CA (US); Pouya Jalalmanesh, Irvine, CA (US)

(21) Appl. No.: 18/419,408

(22) Filed: Jan. 22, 2024

Related U.S. Application Data
(60) Provisional application No. 63/481,365, filed on Jan. 24, 2023.

(51) Int. Cl. A61M 5/145

(52) U.S. Cl. CPC A61M 5/14593; A61M 2202/0486

(57) ABSTRACT

Systems and methods disclosed herein relate to a disease management device which includes at least one or more medication bladders, configured to store fluid and a medication delivery pump, configured to deliver a fluid from one or more medication bladders to a patient. Each medication bladder has at least a rigid portion and a flexible portion, the flexible portion coupled to the rigid portion to create an at least partially sealed enclosure. The rigid portion includes structures, such as channels, configured to facilitate emptying the medication bladder through an output port. On application of pressure from the pump, the flexible portion is configured to generate a pressure against the structures rigid portion, which facilitates emptying of fluid from the medication bladder through the output port.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims the benefit of priority to U.S. Provisional Patent Application No. 63/481,365, filed Jan. 24, 2023.

### FIELD OF INVENTION

The present disclosure relates to medication delivery in minimally invasive disease management systems.

### BACKGROUND

Diabetes is a chronic disease that impacts many individuals, both adults and children. The management of diabetes may include the measurement of glucose within the interstitial space including blood and/or interstitial fluid of a patient and administration of insulin to the patient. A closed loop insulin administration system includes both a sensor to take glucose measurements from the interstitial space including blood and/or interstitial fluid of the patient and an insulin administration device which administers insulin to the patient based on the glucose measurements. Closed loop insulin administration systems allow individuals impacted by diabetes to go about daily life with much less worry about their insulin or glucose levels which can vastly improve a diabetic's quality of life.

### SUMMARY

Various examples of systems, methods, and devices within the scope of the appended claims each have several aspects, no single one of which is solely responsible for the desirable attributes described herein. Without limiting the scope of the appended claims, some prominent features are described herein.

In some aspects, the techniques described herein relate to a disease management device including: one or more medication bladders, configured to store medication, the medication bladder including: a rigid portion, the rigid portion including: a rigid material; an input region configured to facilitate filling the medication bladder with a medication including: an input port; and a plug, coupled to the input port; an output region configured to facilitate emptying the medication bladder of the medication including an output port; a plurality of channels that extend outward from the output region; and a flexible portion, the flexible portion, coupled to the rigid portion for form an at least partially sealed enclosure, including a soft film configured to generate a pressure against the rigid portion; and a medication delivery pump, configured to deliver a medication from one or more medication bladders to a patient.

In some aspects, the techniques described herein relate to a disease management device, wherein the medication includes insulin.

In some aspects, the techniques described herein relate to a disease management device, wherein the medication delivery pump generates a vacuum.

In some aspects, the techniques described herein relate to a medication bladder including: a rigid portion, the rigid portion including: a rigid material; an input port configured to receive a plug; an output port configured to facilitate emptying the medication bladder of the medication; and a plurality of channels that extend outward from a central region of the rigid portion; and a flexible portion coupled to the rigid portion for form an at least partially sealed enclosure, the flexible portion including a soft film configured to generate a pressure to against the rigid portion.

In some aspects, the techniques described herein relate to a medication bladder, wherein the plug is configured to allow a fill device to pass through it to fill the medication bladder with medication.

In some aspects, the techniques described herein relate to a medication bladder, wherein the plug is further configured to self-close after the fill device is removed.

In some aspects, the techniques described herein relate to a medication bladder, wherein the fill device includes a needle.

In some aspects, the techniques described herein relate to a medication bladder, wherein the plug includes silicone.

In some aspects, the techniques described herein relate to a medication bladder, wherein the plug is removeable.

In some aspects, the techniques described herein relate to a medication bladder, wherein the input port is on a section of the rigid portion that protrudes from the medication bladder.

In some aspects, the techniques described herein relate to a medication bladder, wherein the output port is on a section of the rigid portion that protrudes from the medication bladder.

In some aspects, the techniques described herein relate to a medication bladder, wherein the input port is in a central area of the rigid portion.

In some aspects, the techniques described herein relate to a medication bladder, wherein the output port in a central area of the rigid portion.

In some aspects, the techniques described herein relate to a medication bladder, wherein the soft film generates pressure by collapsing against the rigid portion as the medication bladder is emptied.

In some aspects, the techniques described herein relate to a medication bladder, wherein when the soft film is flush with a bulk of the rigid portion, the plurality of channels is configured to continue to allow a flow of medication until the soft film is also flush with the plurality of channels.

In some aspects, the techniques described herein relate to a medication bladder, wherein the rigid portion and the flexible portion are coupled by a process including a laser weld.

In some aspects, the techniques described herein relate to a medication bladder, wherein the rigid portion and the flexible portion are coupled by a process including an ultrasonic weld.

In some aspects, the techniques described herein relate to a medication bladder, wherein the rigid portion and the flexible portion are coupled by a process including an RF weld.

In some aspects, the techniques described herein relate to a medication bladder, wherein the rigid portion and the flexible portion are coupled by a process including overmolding.

In some aspects, the techniques described herein relate to a medication bladder, wherein the rigid portion includes a first surface, wherein the first surface of the rigid portion is not in contact with medication.

In some aspects, the techniques described herein relate to a medication bladder, wherein the rigid portion includes a second surface, wherein the second surface of the rigid portion is in contact with medication.

In some aspects, the techniques described herein relate to a second surface, wherein the second surface is coated with a material to reduce aggregation of the medication.

In some aspects, the techniques described herein relate to a second surface, wherein the material includes silicone dioxide.

In some aspects, the techniques described herein relate to a medication bladder, wherein the rigid material of the rigid portion includes polypropylene.

In some aspects, the techniques described herein relate to a medication bladder, wherein the soft film of the flexible portion includes a thermoplastic elastomer.

In some aspects, the techniques described herein relate to a medication bladder, wherein the medication bladder has a volume of 2.7 mL.

In some aspects, the techniques described herein relate to a medication bladder, wherein the medication bladder has a volume of greater than 2.7 mL.

In some aspects, the techniques described herein relate to a medication bladder, wherein the medication bladder has a volume of less than 2.7 mL.

In some aspects, the techniques described herein relate to a system for delivering medication including: a pump configured to apply a force to one or more medication bladders to deliver medication from the one or more medication bladders to a patient, wherein each medication bladder of the one or more medication bladder includes: a flexible portion configured to apply a pressure against a rigid portion of the medication bladder based on the applied force from the pump to efficiently empty the medication bladder of medication.

In some aspects, the techniques described herein relate to a system, wherein the force applied by the pump is a vacuum.

In some aspects, the techniques described herein relate to a disease management device including: a medication delivery pump configured to deliver a medication from a medication pouch, the medication pouch including: a soft film; and a rigid base including: an inlet port configured to receive medication; a plug configured to block a flow of medication through the inlet port when said inlet port is not receiving medication; an outlet port configured to allow a flow of medication; channels configured to allow the soft film to collapse without blocking the flow of medication through the outlet port.

In some aspects, the techniques described herein relate to an efficiently emptying medication bladder including: a rigid portion, the rigid portion including a rigid material and a plurality of channels extending radially outward from an outlet port of the rigid portion; and a flexible portion coupled to the rigid portion so as to create an at least partially sealed enclosure configured to hold medication for use in a minimally invasive disease management device, the flexible portion including a soft material configured to collapse when medication is pumped out of the medication bladder.

In some aspects, the techniques described herein relate to a method for delivering medication including: identifying a calculated bolus of medication to deliver to a patient; causing a pump to apply pressure to a medication bladder, the pressure based on the calculated bolus; wherein, in response to the applied pressure, the medication bladder is configured to cause an amount of medication to leave an outlet of the medication bladder based on the calculated bolus, wherein at least part of a flexible portion of the medication bladder moves towards a rigid portion of the medication bladder in response to the applied pressure, and wherein the rigid portion includes a plurality of channels configured to facilitate flow of medication in the medication bladder towards the outlet of the medication bladder in the rigid portion.

In some aspects, the techniques described herein relate to a method, wherein the pump is a vacuum pump.

In some aspects, the techniques described herein relate to a method, the method further including, subsequent to the amount of medication leaving the medication bladder, verifying that the calculated bolus has exited the medication bladder.

In some aspects, the techniques described herein relate to a method, wherein the at least part of the flexible portion of the medication bladder moving towards the rigid portion includes compressing the flexible portion against medication in the medication bladder such that at least part of the flexible portion moves closer by a distance that corresponds to the amount of medication that has left the medication bladder.

In some aspects, the techniques described herein relate to a method, the at least part of flexible portion of the medication bladder moving towards the rigid portion includes the at least part of the flexible portion compressing into the plurality of channels of the rigid portion.

In some aspects, the techniques described herein relate to a method, the method further including filling the medication bladder through an inlet of the medication bladder.

In some aspects, the techniques described herein relate to a method, wherein the flexible portion includes a thermoplastic elastomer.

In some aspects, the techniques described herein relate to a method, wherein the rigid portion includes polypropylene.

In some aspects, the techniques described herein relate to a method, wherein the outlet is located in a central region of the rigid portion.

In some aspects, the techniques described herein relate to a method, wherein an inlet of the medication bladder is located in the central region.

In some aspects, the techniques described herein relate to a method, wherein the medication is at least one of insulin or glucagon.

In some aspects, the techniques described herein relate to a method, wherein prior to the amount of medication leaving the medication bladder, the medication bladder is at a first fill state.

In some aspects, the techniques described herein relate to a method, the method further including, subsequent to identifying a calculated bolus, and prior to causing a pump to apply pressure, verifying that the medication bladder at a first fill state holds a first amount of medication that is greater than the calculated bolus.

In some aspects, the techniques described herein relate to a method, wherein subsequent to the amount of medication leaving the medication bladder, the medication bladder is at a second fill state, wherein the medication bladder at the second fill state holds a second amount of medication that is less than the first amount of medication.

In some aspects, the techniques described herein relate to a method, wherein the at least part of the flexible portion moving towards the rigid portion includes the medication bladder moving from the first fill state to the second fill state.

In some aspects, the techniques described herein relate to a method, wherein the second amount of medication is less than the first amount of medication by at least the calculated bolus.

In some aspects, the techniques described herein relate to a method, wherein the second amount of medication is approximately 0% of a maximum volume of the medication bladder.

In some aspects, the techniques described herein relate to a method, wherein the second amount of medication is approximately 5% of a maximum volume of the medication bladder.

In some aspects, the techniques described herein relate to a method, the method further including requesting that the medication bladder be refilled when the first amount of medication is less than the calculated bolus.

In some aspects, the techniques described herein relate to a method, the method further including, requesting that the medication be refilled when the second amount of medication is less than approximately 5% of a maximum volume of the medication bladder.

In some aspects, the techniques described herein relate to a method for delivering medication using a disease management system, wherein the disease management system includes at least a medication bladder and a medication delivery pump, the method including: identifying a calculated bolus of medication to deliver to a patient; causing the medication delivery pump to apply pressure to the medication bladder, the pressure based on the calculated bolus; causing an amount of medication to leave an outlet of the medication bladder based on the calculated bolus, and, in response to the applied pressure, causing at least part of point on a flexible portion of the medication bladder to move towards a rigid portion of the medication bladder in response to the applied pressure, wherein the rigid portion includes a plurality of channels configured to facilitate flow of medication in the medication bladder towards the outlet of the medication bladder in the rigid portion.

In some aspects, the techniques described herein relate to a method, wherein the medication delivery pump is a vacuum pump.

In some aspects, the techniques described herein relate to a method, the method further including, subsequent to the amount of medication leaving the medication bladder, verifying that the calculated bolus has exited the medication bladder.

In some aspects, the techniques described herein relate to a method, wherein the at least part of flexible portion of the medication bladder moving towards the rigid portion includes compressing the flexible portion against medication in the medication bladder such that at least part of the flexible portion moves closer by a distance that corresponds to the amount of medication that has left the medication bladder.

In some aspects, the techniques described herein relate to a method, the at least part of flexible portion of the medication bladder moving towards the rigid portion includes the at least part of the flexible portion compressing into the plurality of channels of the rigid portion.

In some aspects, the techniques described herein relate to a method, the method further including filling the medication bladder through an inlet of the medication bladder.

In some aspects, the techniques described herein relate to a method, wherein the flexible portion includes a thermoplastic elastomer.

In some aspects, the techniques described herein relate to a method, wherein the rigid portion includes polypropylene.

In some aspects, the techniques described herein relate to a method, wherein the outlet is located in a central region of the rigid portion.

In some aspects, the techniques described herein relate to a method, wherein an inlet of the medication bladder is located in the central region.

In some aspects, the techniques described herein relate to a method, wherein the medication is at least one of insulin or glucagon.

In some aspects, the techniques described herein relate to a disease management device including: a pump configured to deliver fluid to an injection site of a patient; one or more medication bladders configured to store the fluid prior to delivery to the injection site of the patient, each of the one or more medication bladders including: a rigid portion coupled to a flexible portion to form an at least partially sealed enclosure, the rigid portion including: an outlet port configured to allow an outward flow of the fluid from the at least partially sealed enclosure towards the pump, wherein the flexible portion includes a flexible material configured to change shape so as to facilitate movement of the fluid within the at least partially sealed enclosure towards the outlet port of the rigid portion.

In some aspects, the techniques described herein relate to a disease management device, wherein the rigid portion further includes a first interior surface, and wherein the flexible portion further includes a second interior surface, and wherein the first interior surface and second interior surface further include an anti-aggregation coating, wherein the anti-aggregation coating facilitates the movement of fluid within the at least partially sealed enclosure towards the outlet port of the rigid portion.

In some aspects, the techniques described herein relate to a disease management device, the rigid portion further including: an inlet port configured to facilitate access to the partially sealed enclosure for filling the medication bladder with the fluid.

In some aspects, the techniques described herein relate to a disease management device, wherein the flexible material of the flexible portion is configured to change shape at least by compressing against the rigid portion, wherein compression of the flexible material against the rigid portion facilitates the movement of fluid within the at least partially sealed enclosure towards the outlet port of the rigid portion.

In some aspects, the techniques described herein relate to a disease management device, wherein the compression is caused by an application of an external pressure to the respective medication bladder of the one or more medication bladders.

In some aspects, the techniques described herein relate to a disease management device, wherein the disease management device further includes a pressure applicator configured to apply external pressure to at least one of the one or more medication bladders.

In some aspects, the techniques described herein relate to a disease management device, wherein the pressure applicator is in communication with the pump, and the applied external pressure is based on a force received from the pump.

In some aspects, the techniques described herein relate to a disease management device, wherein the outlet port is centered with respect to the rigid portion.

In some aspects, the techniques described herein relate to a disease management device, wherein the flexible portion further includes one or more guiding surfaces, wherein the guiding surfaces include at least one of a curved surface or an inclined surface, and wherein the one or more guiding surfaces are configured to facilitate the movement of fluid within the partially sealed enclosure towards the outlet port of the rigid portion.

In some aspects, the techniques described herein relate to a disease management device, wherein the rigid portion includes: a first curved surface of the one or more guiding surfaces extending from an edge of the rigid portion to a first inclined surface of the one or more guiding surfaces, wherein the edge is between the rigid portion and the flexible portion, wherein the first inclined surface is configured to facilitate the movement of fluid within the at least partially scaled enclosure towards a second curved surface, wherein the second curved surface is located at a center of the rigid portion, and wherein the second curved surface includes the outlet port.

In some aspects, the techniques described herein relate to a disease management device, the rigid portion further including: an interior surface including: a plurality of channels configured to extend from at least one edge of the rigid portion towards the outlet port.

In some aspects, the techniques described herein relate to a disease management device, wherein the rigid portion further includes one or more protrusions, wherein each channel of the plurality of channels forms a depression within a protrusion of the one or more protrusions, and wherein the depression is configured to facilitate movement of fluid within the at least partially sealed enclosure towards the outlet port.

In some aspects, the techniques described herein relate to a disease management device, wherein the rigid portion further includes one or more guiding surfaces, including at least one of a curved surfaces or an inclined surface, wherein the one or more guiding surfaces apply contours to the rigid portion to facilitate the movement of fluid within the partially sealed enclosure towards the outlet port of the rigid portion, and wherein the plurality of channels follow the contours of the rigid portion.

In some aspects, the techniques described herein relate to a disease management device, wherein the flexible material of the flexible portion is configured to change shape at least by compressing against the plurality of channels of rigid portion, wherein compression of the flexible material against the plurality of channels of the rigid portion facilitates the movement of fluid with the at least partially sealed enclosure towards the outlet port of the rigid portion.

In some aspects, the techniques described herein relate to a disease management device, wherein the flexible material of the flexible portion is configured to change shape in response to a vacuum applied by the pump.

In some aspects, the techniques described herein relate to a disease management device, wherein the rigid portion further includes: one or more walls; one or more edges; a central region including the outlet port; a top surface; and one or more protruding structures, wherein the one or more protruding structures extend vertically with respect to a major plane of the top surface, wherein the one or more protruding structures extend from the at least of the one or more walls or one or more edges to the central region.

In some aspects, the techniques described herein relate to a disease management device, wherein the one or more protruding structures vary in height with respect to a major plane of the top surface.

### BRIEF DESCRIPTION

These and other features, aspects, and advantages of the present application are described with reference to drawings of certain examples, which are intended to illustrate, but not limit, the present disclosure. It is to be understood that the attached drawings are for the purpose of illustrating concepts disclosed in the present application and may not be to scale:

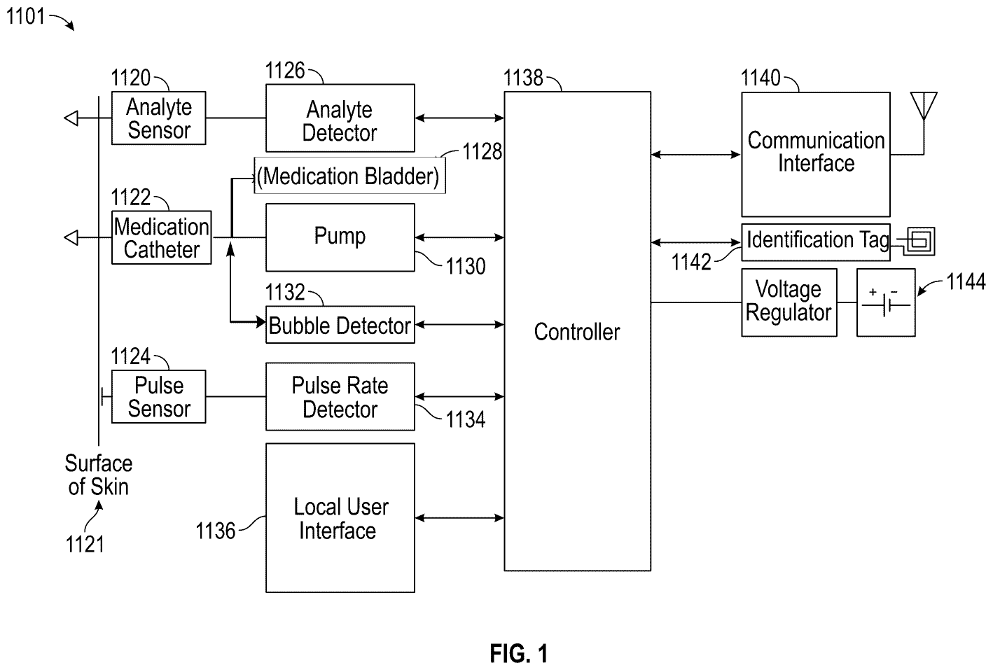

FIG. 1 illustrates an example disease management system that may be part of a disease management environment.

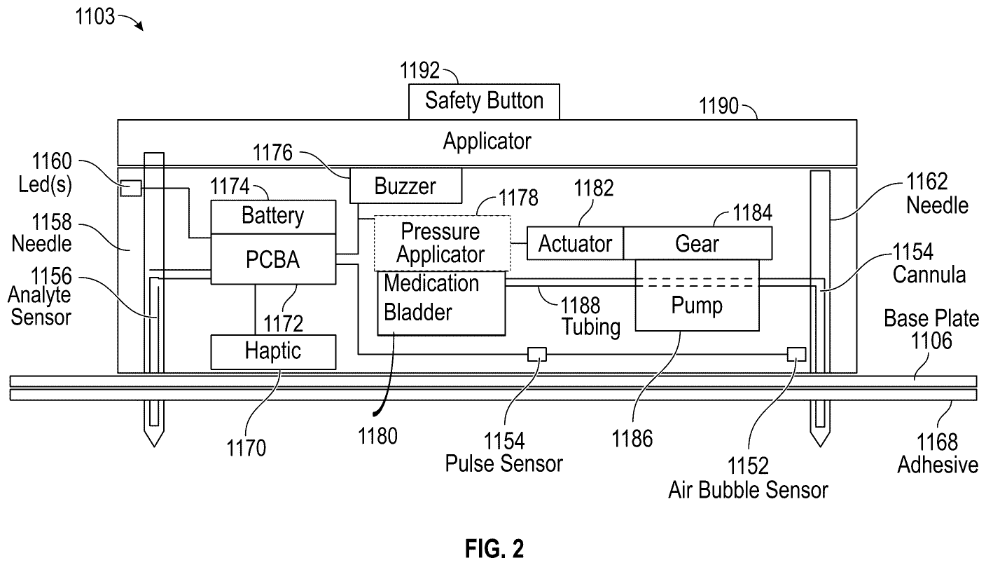

FIG. 2 illustrates an example implementation of a disease management system.

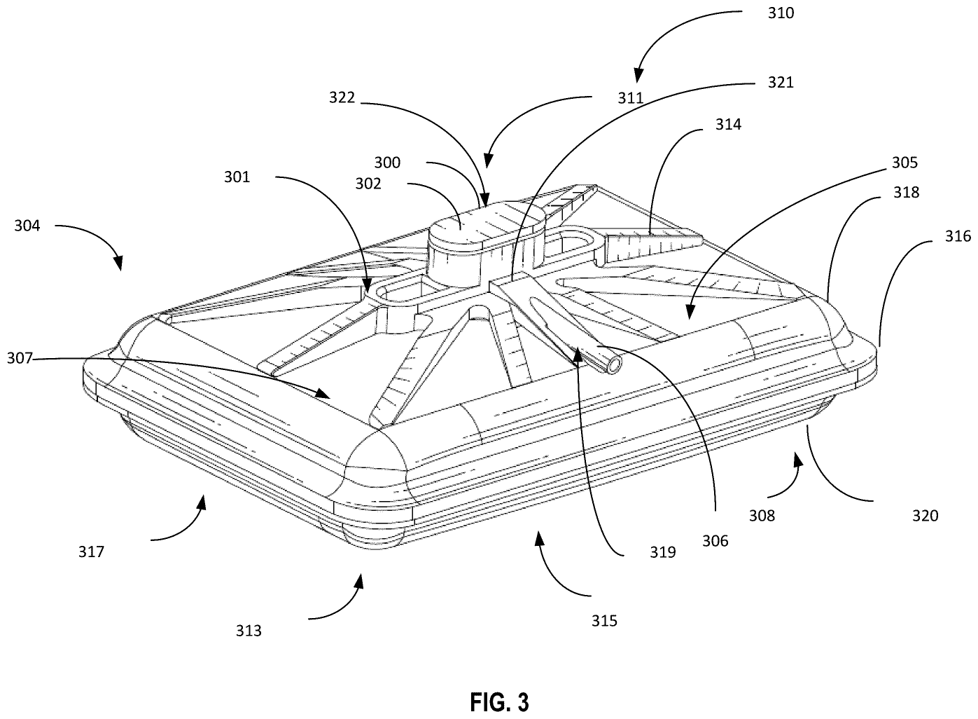

FIG. 3 illustrates an example implementation of an efficiently emptying medication bladder.

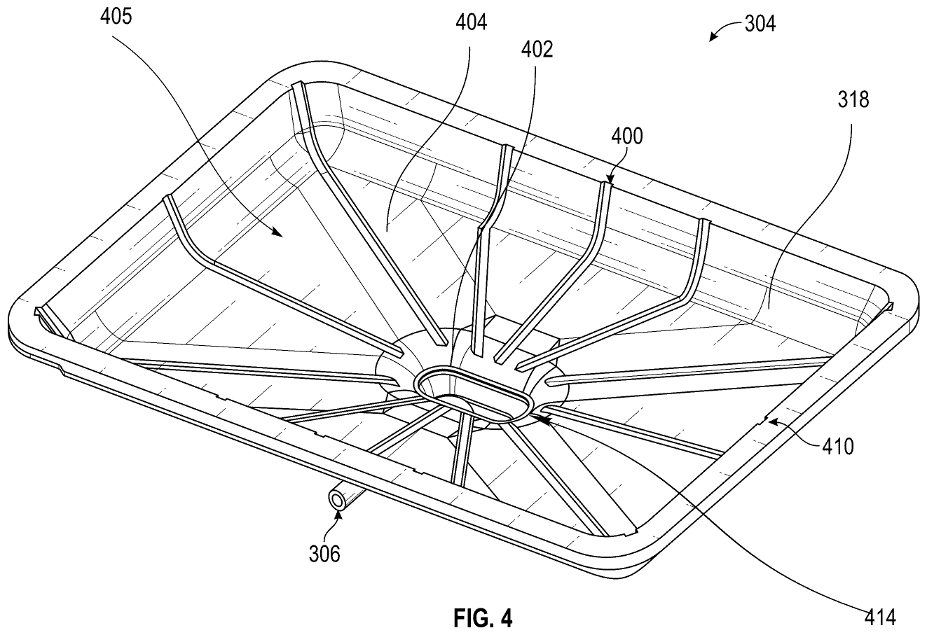

FIG. 4 illustrates an example implementation of a rigid portion of an efficiently emptying medication bladder.

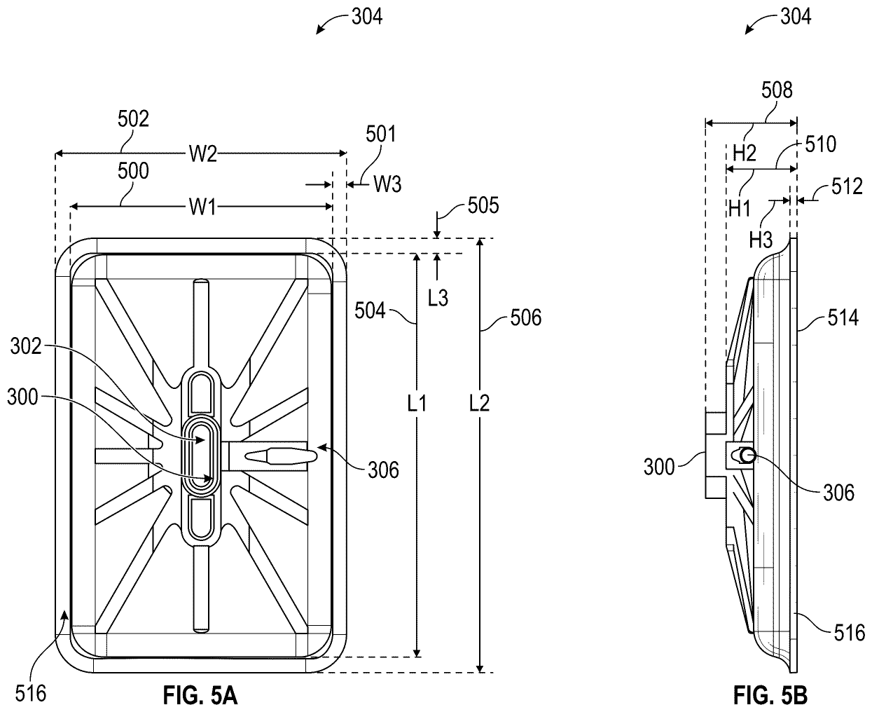

FIGS. 5A-B illustrate views of an example implementation of a rigid portion of an efficiently emptying medication bladder.

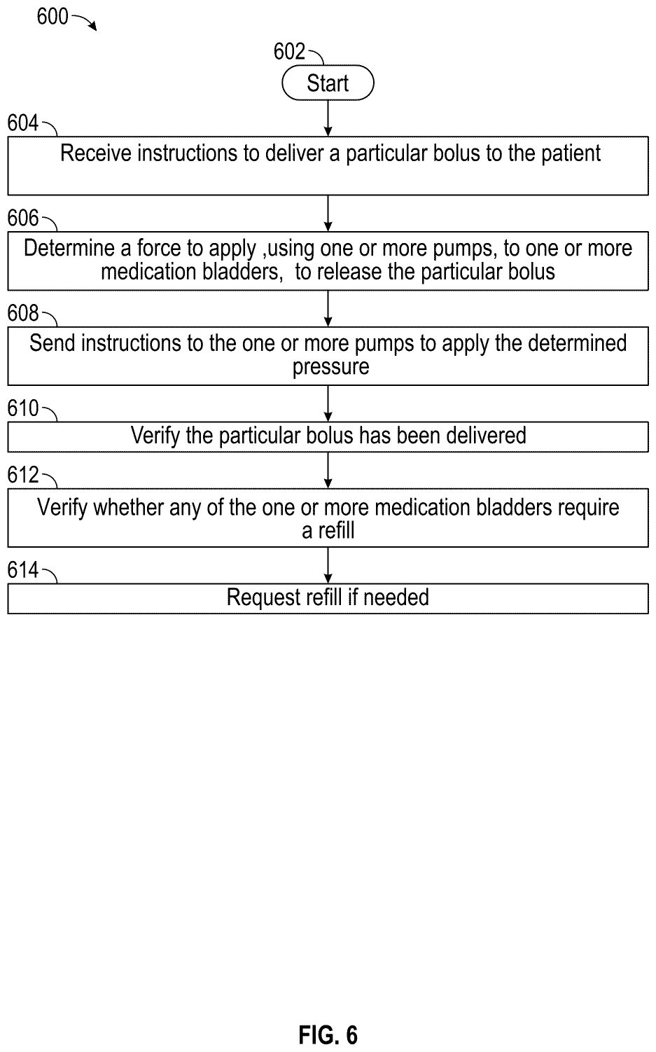

FIG. 6 illustrates an example routine that may be performed by a disease management system.

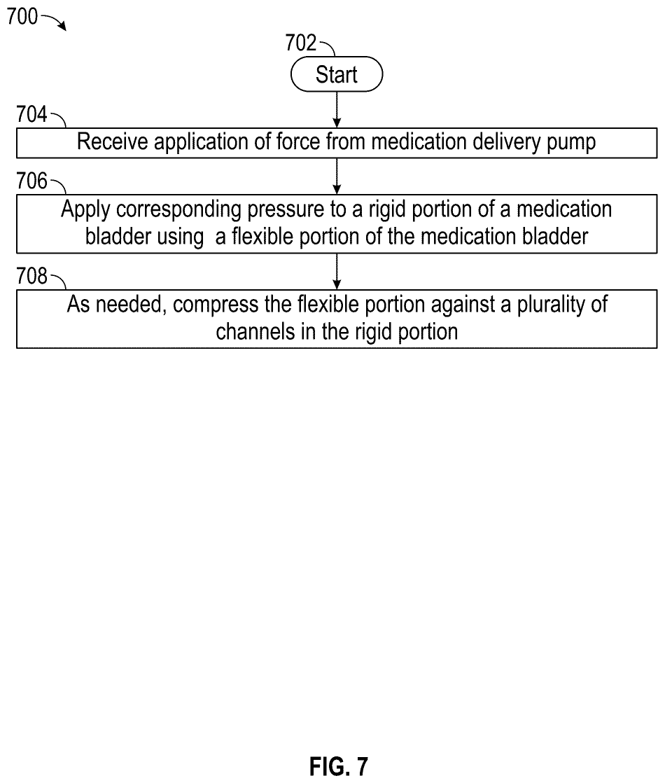

FIG. 7 illustrates an example process of releasing medication from an embodiment of a medication bladder.

### DETAILED DESCRIPTION

Although certain preferred examples and examples are disclosed below, inventive subject matter extends beyond the specifically disclosed examples to other alternative examples and/or uses and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular embodiment. Thus, for example, various examples may be carried out in a manner that achieves or optimizes one advantage or group of advantages as taught herein without necessarily achieving other aspects or advantages as may also be taught or suggested herein.

Certain chemical substances, such as medications for management of diabetes or other physiological condition, can be administered by a minimally invasive implant, such as a medication delivery pump or the like. It is important that such systems are consistent and accurate. Over delivering or under delivering medication may lead to adverse health consequences. With respect to delivery of insulin, for example, delivering too much insulin to a patient may result in hypoglycemia, which can lead to loss of consciousness, comas, or even death. Delivering too little insulin may lead to hyperglycemia which may cause significant damage to internal organs such as the heart.

Aspects of the present disclosure provide a medication bladder that can advantageously reliably release fluid including chemical substances, such as medication for management of diabetes or other physiological condition. The medication bladder may reliably release the fluid at different levels of fullness of the bladder and reduce medication waste by facilitating improved emptying of the medication bladder. For example, the medication bladder may include a rigid portion and a flexible portion. The rigid portion may include at least an outlet port and a plurality of channels the facilitate release of fluid from the medication in response to the applied force. In some examples, the flexible portion may apply force through application of pressure against the medication in the medication bladder. This may facilitate the flow of fluid through the outlet on the rigid portion of the medication bladder.

In some aspects, the medication bladder may be in communication with a pump. The pump may apply a force, such as a vacuum force, to the medication bladder. This may cause the flexible portion of the medication bladder to compress against the channels on the rigid portion, which may facilitate the flow of the remaining fluid from the outlet. The interior surfaces of the medication bladder may be coated in a material to mitigate aggregation of the fluid. This coating may further facilitate the flow of fluid from the medication bladder at least by reducing the medication remaining on the interior surfaces.

### Components of an Example Disease Management System

FIG. 1 shows a block diagram of an example disease management system 1101 including a medication bladder 1128, that may be in accordance with some aspects of the present disclosure. An example disease management system 1101 and/or features thereof is described with respect to U.S. Pat. Pub. No. 2021/0236729, filed Jan. 28, 2021, titled “REDUNDANT STAGGERED GLUCOSE DISEASE MANAGEMENT SYSTEM,” which is incorporated herein in its entirety.

In some examples, the disease management system 1101 may be part of a disease management environment, such as described above. A disease management system 1101 may be configured to measure one or more physiological parameters of a patient, such as pulse, skin temperature, or other values. The disease management system 1101 may further be configured measure one or more analytes present in the blood of a patient, such as glucose, lipids, or other analytes. Additionally, or alternatively, the disease management system 1101 may administer fluid for patient treatment. The fluid may include one or more chemical substances. For example, the fluid may include, but is not limited to including, medication (e.g., as insulin, glucagon, another medication, some combination thereof) and/or various additives. As used herein, additives may refer to chemical substances used to impact performance of the medication. For example, additives may impact how the medication is absorbed in a manner that impacts the time it takes for the medication to take effect. Additives may also impact how long the medication is viable. Additives may include, but are not limited to preservatives, stabilizers, surfactants, buffers, antimicrobials, zinc, another chemical substance, or some combination thereof. Example chemical substances included in the fluid are described and/or illustrated in U.S. Pat. Pub. No. 2022/0096603 filed Sep. 28, 2021, titled “INSULIN FORMULATIONS AND USES IN INFUSION DEVICES,” which is incorporated herein in its entirety.

In some examples, the disease management system 1101 may be modular. As used herein, “modular” may mean that at least some of the components of the disease management system 1101 may be separable from other components of the disease management system 1101. For example, the disease management system 1101 may be configured to supply power, as needed, to the components illustrated in FIG. 1. At least some of the components illustrated in FIG. 1 may be a modules removably coupled to the disease management system 1101 through one or more electrical connectors, not shown. For example, the controller 1138 may be a reusable module. When a patient needs to replace the disease management system 1101, the patient can remove the controller 1138 from a first disease management system 1101 and couple the controller module 1138 to a second disease management system 1101. The controller 1138 may then control components of the second disease management system 1101. As another example, the controller 1138 of the first disease management system 1101 may malfunction, and a new controller 1138 may be placed in the first disease management system 1101 by a patient or caregiver. The new controller 1138 may then control components of the first disease management system 1101.

While the example above describes the controller 1138 as a module removably coupled to the disease management system 1101, this is not intended to be limiting. Other components of the disease management system 1101 may also be modules removably coupled to the disease management system 1101. For example, the medication bladder 1128 may be replaceable and/or removable from the disease management system 1101. When removed from the disease management system 1101, the medication bladder 1128 may be renewed (such as cleaned and/or refilled with medication) and/or a new medication bladder 1128 may be inserted in place of the medication bladder 1128 into the disease management system 1101.

In some examples, a disease management system 1101 may be configured to communicate with one or more hardware processors that may be external to the disease management system 1101, such as a cloud based processor or user device. A disease management system 1101 may include an identification tag 1142, such as a near field communication tag or a radio frequency identification (RFID) tag to support authentication and pairing with a user device (for example, smart phone or smart watch), a communication interface 1140. The communication interface 1140 may support Bluetooth communication with additional disease management systems or devices, and Bluetooth communication with a paired user device running an associated control application. The communication interface 1140 may also support communication with other communication protocols including, but not limited to, Transmission Control Protocol/Internet Protocol (TCP/IP), Message Queuing Telemetry Transport (MQTT), or Wi-Fi.

To support case of use and safe interaction with the patient, the system may incorporate user input through a tap-detecting accelerometer and provide feedback via an audio speaker, haptic vibration, and/or optical indicators. The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

As illustrated in FIG. 1, a controller 1138 of the disease management system 1101 may be configured to communicate and control one or more components of the disease management system 1101. The controller 1138 may include one or more hardware processors, such as a printed circuit board (PCB) or the like. The controller 1138 may be configured to communicate with peripheral devices or components to support the accurate measurement of physiological parameters and blood analytes, such as patient pulse, temperature, and blood glucose, using detector electronics. The controller 1138 may subsequently calculate dose or receive a calculated dose value and administer chemical substance(s), such as insulin, by actuation of an actuated pump. The controller 1138 may record device activity and transfer the recorded data to non-volatile secure memory space. In some examples, a user (e.g., a manufacturer, caregiver, or patient) may determine to no longer use the disease management system 1101. For example, the user may want to replace the disease management 1101. In further examples, the controller 1138 can be configured to lock operation, and create a data recovery module to permit authenticated access to the recorded data if needed.

A disease management system 1101 may include an analyte sensor 1120. The analyte sensor 1120 may be configured to detect analytes in the patient's blood. For example, an analyte sensor 1120 can include a glucose sensing probe configured to pierce the surface of the skin 1121. In some examples, a disease management system 1101 may include a plurality of analyte sensors 1120 to detect one or more analytes. In some examples, an analyte sensor 1120 may be configured to detect a plurality of analytes. Sensed analytes may include, but are not limited to, glucose, insulin, and other analytes. An analyte sensor 1120 may be configured to communicate with an analyte detector 1126. The analyte detector 1126 may be configured to receive a signal of one or more analyte sensors 1120 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller.

A disease management system 1101 may include a medication catheter 1122. The medication catheter 1122 may be configured to administer medication, including, but not limited to insulin, to the patient. The medication catheter 1122 may receive fluid including chemical substances such as medication from at least one medication bladder 1128. The medication bladder 1128 may be configured to hold and/or release fluid including chemical substances, such as medication and/or additives, to be administered. The medication bladder 1128 may be configured to hold fluid for a prolonged period, such as 1 day, 3 days, 6 days, or more. The time period for storage may impact the size of the medication bladder 1128.

As discussed, the medication bladder 1128 may be configured to hold fluid including certain medications, such as insulin. In some examples, a disease management system 1101 may include a plurality of medication bladders 1128 for one or more reservoirs of the same or different fluid. The same fluids may include the same chemical substances (e.g., medication, additives, and the like). Different fluids may include different chemical substances (e.g., medication, additives, and the like). In some examples, a disease management system 1101 may be configured to mix fluids from multiple medication bladders 1128 prior to administration to the patient. A pump 1130 may be configured to cause the fluid to be administered from the bladder 1128 to the patient through the medication catheter 1122. The pump 1130 can incorporate any of the features described with respect to any of the devices described and/or illustrated in U.S. Pat. Pub. No. 2023/0115397, filed Aug. 4, 2022, titled “MEDICATION DELIVERY PUMP FOR REDUNDANT STAGGERED GLUCOSE SENSOR INSULIN DOSAGE SYSTEM,” which is incorporated herein in its entirety.

In some examples, a disease management system 1101 may be configured to include a plurality of physiological sensors, such as a physiological sensor 1124. The physiological sensor 1124 may include a pulse rate sensor, temperature sensor, pulse oximeter, the like or a combination thereof. Each physiological sensor 1124 may be configured to communicate with a physiological detector 1134. The physiological detector 1134 may be configured to receive a signal or signals of the physiological sensor 1124. The physiological detector 1134 may also be configured to measure or determine a physiological value from the signal. Determinations and measurements by the physiological detector 1134 may be responsive to signals from the controller 1138. The physiological detector 1134 may additionally, or alternatively, communicate the physiological values to a destination, such as controller 1138.

A disease management system 1101 may include one or more local user interfacing components 1136. For example, a local user interfacing component 1136 may include, but is not limited to one or more optical displays, haptic motors, audio speakers, and user input detectors. In some examples, an optical display may include an LED light configured to display a plurality of colors. In some examples, an optical display may include a digital display of information associated with the disease management system 1101, including, but not limited to, device status, medication status, patient status, measured analyte or physiological values, the like or a combination thereof. In some examples, a user input detector may include an inertial measurement unit, tap detector, touch display, or other component configured to accept and receive user input. In some examples, audio speakers may be configured to communicate audible alarms related to device status, medication status user status, the like or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include a communication interface 1140 with one or more communication components. A communication component can include, but is not limited to, one or more radios configured to emit Bluetooth, cellular, Wi-Fi, or other wireless signals. In some examples, a communication component can include a port for a wired connection. Additionally, a disease management system 1101 may include an identification tag 1142 to facilitate in communicating with one or more hardware processors. The one or more communication components of the communication interface 1140 and identification tag 1142 may be configured to communicate with the controller 1138 in order to send and/or receive information associated with the disease management system 1101. For example, a controller 1138 may communicate medication information and measured values through the one or more components of communication interface 1140 to an external device. Additionally, the controller 1138 may receive instructions associated with measurement sampling rates, medication delivery, or other information associated with operation of the management system 1101 through the one or more components of communication interface 1140 from one or more external devices.

A disease management system 1101 may include one or more power components 1144. The power components 1144 may include, but are not limited to, one or more batteries and power management components, such as a voltage regulator. Power from the one or more power components 1144 may be accessed by the controller and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the RTC (or real time clock) and alarms to wake the system and take a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake up mode. The wake up mode may be triggered by an NFC device and allow the system to pair with an external device with, for example, Bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculation, are performed and data is communicated to an external device and/or the cloud. In another example, a disease management system 1101 may include a pump mode where the system is configured to enter a higher power mode where the pump draws power to deliver medication to the patient.

While the description above describes the medication bladder 1128 as part of a disease management system 1101, this is not intended to be limiting. For example, the medication bladder 1128 may be included in a standard insulin pump. In further examples, the medication bladder 1128 may be removeable. When removed the medication bladder 1128 may be cleaned, and/or refilled.

In some examples, a disease management system 1101 may include one or more connector test points 1146. The connecter test points may be configured to aid in programming, debugging, testing or other accessing of the disease management system 1101. In some examples, connector test points 1146 may include, for example, a GPIO spare, UART receiver or transmitter, the like or a combination thereof.

FIG. 2 illustrates an example implementation of a disease management system 1103 and applicator 1190 for applying a disease management system 1103 to a patient. Disease management system 1103 can include any one or more of the features discussed above with respect to the disease management system 1101 in addition or in the alternative to the features described below. In the illustrated example, an applicator 1190 may be configured to couple to the disease management system 1103. In some examples, an applicator 1190 may include a safety button 1192 for release or other interaction with the applicator 1190. In the illustrated example, a disease management system 1103 may include one or more LEDs 1160 that may be configured to output information using one or more of color, frequency, and length of display. In some examples, the disease management system 1103 may include a buzzer 1176, haptic actuator 1170, or other feedback mechanism, such as a speaker to output information to the patient, such as an alarm. In some examples, a disease management system 1103 may include a battery 1174, controller 1172. In some examples, a disease management system 1103 may include aspects of a medication administration system, such as a medication bladder 1180, a pressure applicator 1178, actuator 1182, pump gears 1184, and a pump 1186. The medication bladder 1180 may include the components and/or functions described above with respect to medication bladder 1128 of FIG. 1. In some examples, the pressure applicator 1178 may be a component of the pump 1186. In other examples, the pressure applicator 1178 may be separated from the pump 1186.

As discussed with respect to FIG. 1, disease management system 1103 may be modular. The disease management system 1103 may be configured to supply power through base plate 1106. At least some of the components illustrated in FIG. 2 may be a modules removably coupled to the disease management system 1103 through one or more electrical connectors, not shown, on the base plate 1106. For example, the medication bladder 1180 may be replaceable and/or removable from the disease management system 1103 through the base plate 1106. When removed from the, the medication bladder 1180 may be renewed (such as cleaned and/or refilled with medication) and/or a new medication bladder 1180 may be inserted in place of the medication bladder 1180 into the disease management system 1103.

The pressure applicator 1178 may apply a pressure to the medication bladder 1180 in response to instructions from a controller, such as controller 1138 of FIG. 1. The controller may determine to deliver a specified bolus. The controller may then determine instructions pressure applicator 1178 to provide the specified bolus, and provide these instructions to the pressure applicator 1178.

In response to instructions, the pressure applicator 1178 may provide a positive or negative pressure to the medication bladder 1180. The pressure applied by the pressure applicator 1178 may facilitate the release of fluid including chemical substances from the medication bladder 1180. In some implementations, to generate the pressure, the pressure applicator 1178 may generate a force for application to the medication bladder 1180. For example, the pressure applicator 1178 may be a plunger or other actuator. Alternately, the pressure applicator 1178 may be the same as the pump 1186. For example, the pump 1186 may be configured to apply a negative pressure to the medication bladder 1180 to withdraw medication from the medication bladder 1180. Additionally, or alternatively, another device may be configured to apply a positive or negative pressure to the medication bladder 1180. In some examples, both the pump 1186 and another device may work together to cause medication to transfer from the medication bladder 1180.

In some examples, in response to the force or pressure, the medication bladder 1180 may release fluid including chemical substances through an outlet port, as will be described in more detail in FIG. 3. The pump 1186 may subsequently release the fluid including chemical substances into a patient through an injection site in accordance with a specified dosage. For example, the fluid including chemical substances may be provided through needle 1158 and/or needle 1162.

Additionally, or alternatively, the medication bladder 1180 may release fluid including chemical substances to a mixing area, not shown. The mixing area may combine chemical substances, such as medications, from one or more medication bladders 1178. The mixed fluid may then be provided to the patient through an injection site in accordance with a specified dosage. For example, the fluid including chemical substances may be provided through needle 1158 and/or needle 1162.

In some examples, a disease management system 1103 may include one or more needles 1158 that may include one or more analyte sensors (such as a glucose sensor) 1156. In some examples, a disease management system 1103 may include one or more needles 1162 that may include one or more cannulas 1164 configured to administer medication to the patient. In some examples, a disease management system 1103 may include an air bubble sensor 1152 configured to detect the presence of air bubbles in the medication prior to delivery to the patient. In some examples, the disease management system 1103 may include one or more physiological sensors 1154, such as a non-invasive physiological sensor including but not limited to a pulse sensor. In some examples, the disease management system 1103 may include a base plate 1106 and an adhesive layer 1168 below the base plate 1106 to provide adhesion of the disease management system 1103 to the patient's skin. As described below, a housing of the disease management system 1103 may consist of a combination of flexible and rigid material so as to both provide support for the components of the disease management system 1103 and allow conforming, at least in part, of the disease management system 1103 to the skin of the patient.

The adhesive layer 1168 may be configured to provide adhesion for a prolonged period. For example, the adhesive layer 1168 may be configured to adhere the disease management system 1103 to the skin of a patient for a period of 1 day, 3 days, 6 days, or more or fewer days or hours. In some examples, the adhesive layer may be configured to have an adhesive force sufficient to prevent accidental removal or movement of the disease management system 1103 during the intended period of use of the disease management system 1103. In some examples, the adhesive layer 1168 may be a single layer of adhesive across at least a portion of a surface the disease management system 1103 that is configured to interface with the patient. In some examples, the adhesive layer 1168 may include a plurality of adhesive areas on a surface of the disease management system 1103 that is configured to interface with the patient. In some examples, the adhesive layer 1168 may be configured to be breathable, adhere to the patient's skin after wetting by humidity or liquids such as tap water, saltwater, and chlorinated water. A thickness of the adhesive may be, for example, in a range of 0.1 to 0.5 mm or in a range of more or less thickness.

In some examples, a needle 1158, 1162 may be inserted at different depths based on a patient age, weight, or other parameter. For example, a depth of insertion of a medication cannula may be approximately 3 mm for 7 to 12 year olds. In another example, a depth of insertion of a medication cannula may be approximately 4 mm for 13 year olds and older. In another example, a depth of insertion of a medication needle may be approximately 4 to 4.5 mm for 7 to 12 year olds. In another example, a depth of insertion of a medication needle may be approximately 5 to 5.5 mm for 13 year olds and older. In another example, a depth of insertion of an analyte sensor may be approximately 3 mm for 7 to 12 year olds. In another example, a depth of insertion of an analyte sensor may be approximately 4 mm for 13 year olds and older. In another example, a depth of insertion for a needle associated with an analyte sensor may be approximately 4 to 4.5 mm for 7 to 12 year olds. In another example, a depth of insertion for a needle associated with an analyte sensor may be approximately 5 to 5.5 mm for 13 year olds and older. However, other values or ranges for any of the inserted components are also possible.

While the description above describes the medication bladder 1180 as part of a disease management system 1103, this is not intended to be limiting. For example, the medication bladder 1180 may be included in a standard insulin pump. In further examples, the medication bladder 1180 may be removeable. When removed the medication bladder 1180 may be cleaned, and/or refilled.

### Example Medication Bladder

FIG. 3 illustrates an example of an exterior side of a rigid portion 304 of an example medication bladder 310 that may be part of a disease management, such as disease management system 1101 of FIG. 1 and disease management system 1103 of FIG. 2. FIG. 4 illustrates a view of an interior side of a rigid portion 304 showing the interior surface 405 of rigid portion 304. As illustrated, the interior surface 405 may be on an opposing side of an exterior surface 305. For example, the interior surface 405 may be on an interior side of rigid portion 304, such as within an enclosure formed by rigid portion 304 and flexible portion 308, where the interior surface 405 forms at least part of the interior bounds of the enclosure.

In some examples, the medication bladder 310 may include the features described above with respect to medication bladder 1128 of FIG. 1 and/or medication bladder 1180 of FIG. 2 in addition to features described below. However, other implementations in other medication delivery and/or storage systems are also possible. In some implementations, such as in the illustrated example of FIG. 3, the medication bladder 310 may include at least two exterior housing components configured to at least partially couple. The exterior housing components, when coupled, may be configured to form a bladder, pouch, or other enclosure for holding one or more fluids, such as a medication for administration to a wearer of a medication management system, including but not limited to a system such as described with reference to FIG. 1 or 2 or another medication delivery system.

The at least two exterior housing components may include at least one rigid portion 304 and at least one flexible portion 308. The at least one rigid portion 304 may be configured to maintain shape under a threshold amount of pressure and the at least one flexible portion 308 may be configured to move, change shape, deflate, or otherwise modify its shape or orientation when exposed to at least a minimum threshold positive or negative pressure. The material of the rigid portion 304 may be a rigid material including, but not limited to, ABS, polypropylene (PP), polycarbonate (PC) or metal, or some combination thereof. The material of the flexible portion 308 may be a soft material including, but not limited to, thermoplastic elastomer (TPE), silicone, or some combination thereof. The flexible portion 308 may be in the form of a soft film of the selected material or materials. The interior surfaces of the rigid portion 304 and flexible portion 308 may be coated with an anti-aggregation coating including, but not limited to silicone dioxide or zinc oxide.

In some examples, the at least one rigid portion 304 may have at least one input port 300 with at least one inlet seal 302 and at least one output port 306. The at least one rigid portion 304 may include a geometry having at least one external surface 305, at least one internal surface 405, at least one wall 318, and at least one lip or edge 316. The geometry of the rigid portion 304 may be or include guiding structures such as curved surfaces or inclined planes, which may facilitate the movement of fluid to the output port or output ports 306.

As shown in FIG. 3, the exterior surface 305 of the rigid portion 304 may be on an exterior side of the enclosure formed by rigid portion 304 and flexible portion 308. The at least one external surface 305 of the rigid portion 304 may include at least one top surface 307, and at least one structure 314. The top surface 307 may form an approximately flat surface. The top surface 307 may additionally or alternatively for an inclined surface. The inclined surface may tilt from an edge, such as edge 316, towards a central region 311.

In some examples, the at least one structure 314 may be configured to extrude, couple to, or otherwise extend from the at least one top surface 307. The at least one structure 314 may be configured to provide a supporting structure for the rigid portion 304, improving the strength and/or rigidity of the rigid portion 304. In some examples, the at least one structure 314 may be configured to support one or more areas of the rigid portion 304 associated with at least one channel 400 on an interior of the rigid portion 304, such as illustrated in FIG. 4. In further examples, the channel 400 may be embedded in the rigid structure to form a depression with respect to interior surface 405. The at least one structure 314 may be configured to additionally or alternatively provide support for other components of a rigid portion 304, such an input port 300 and/or output port 306.

In some examples, the at least one structure 314 may include a plurality of structures, such as 6, 8, 10, or more structures or structural components. The structures 314 may protrude from the top surface 307. The at least one structure 314 may be configured to extend from at least a portion of an edge 316 or wall 318 of the rigid portion 304 towards a central region 311 of the rigid portion 304, which may include an input port 300 or other structure 301 surrounding and/or supporting an input port 300, which may or may not form part of the at least one structure 314. The location of each structure of 314 on the top surface 307 may correspond to the location of at least one channel 400 of FIG. 4.

The at least one structure 314 may vary in size and/or shape across the geometry of the rigid portion 304. For example, the at least one structure 314 may be configured to increase in height and/or change shape or profile between an edge 316 and/or wall 318 towards the central region 311. In some examples, the at least one structure 314 may be configured to have a different shape and/or more or fewer structures may be placed on the area of the rigid portion 304 based on a location of the rigid portion 304, such as, where the rigid portion 304 has an approximately rectangular top-down profile, nearer a corner 313, major edge 315, or minor edge 317.

A central region 311 may include at least one structure configured to extend perpendicularly from a major plane of the at least one external surface 305 of the rigid portion 304. The central region 311 may include a cavity or central depression 402 on a bottom or interior side of a rigid portion 304, as illustrated in FIG. 4. Advantageously, the form of the central region 311 may facilitate gathering and/or flow of medication from an interior of a medication bladder 310 towards an output region 319. As will be described herein, the rigid portion 304 may include one or more additional features that can help facilitate movement of medication towards this central region 311 and/or depression 402 of the central region 311, such as a plurality of channels 400 on an interior surface 405, a contour or shape of the interior surface 405, a coating of the interior surface 405, and/or other features as described herein.

With continued reference to FIG. 3, the central region 311 may include an input region 322 and/or an output region 319. The input region 322 may include an inlet port 300 and/or associated inlet seal 302. The inlet port 300 may be configured to have an opening at a center of the central region 311 such that the inlet port 300 can be accessed from a top of the rigid portion 304. The inlet seal 302 may be configured to at least partially seal the inlet port 300, as discussed herein.

The central region 311 may further include, in some implementations, at least a part of an output region 319, such as a lateral or otherwise oriented opening 321 of an output region 319. In some examples, the output region 319 may be offset from the central region 311 of the rigid portion 304. In some examples, the output region 319 may be oriented to output fluid at a lateral location, such as at, near, or above an edge 316. In some examples, the output region 319 may be configured to extend from a central region 311 towards an edge 316 of the rigid portion 304. However, other configurations are also possible. Additionally, or alternatively, the output region 319 could be located on a flexible portion 308 of the medication bladder 310. The output region 319 may additionally, or alternatively, protrude upward from the at least one top surface 307 and/or bulk or planar portion of the rigid portion 304, such as illustrated in in FIG. 3.

The output region 319 may be configured to facilitate evacuating medication from the medication bladder 310. The one or more output ports 306 may serve as outlets to allow medication to exit the medication bladder. For example, the output region 319 may include one or more output ports 306. An output port 306 may facilitate the flow of medication by providing an opening through which the medication can leave the output region 319 and the medication bladder 310. The one or more output ports 306 may include, but are not limited to a tube or cannula extending from a portion of the rigid portion 304, such as a lateral or otherwise oriented opening 321 in a central region 311 of the rigid portion 304. The one or more output ports 306 may be composed of one or more materials, which may include, but is not limited to metal or silicone. In addition, the one or more output ports may contribute to facilitating the flow of medication through the output region 319 and out of the medication bladder 310.

In some examples, one or more output ports 306 may be used together to increase the rate of the flow of medication through the output region 319 and out of the medication bladder 310. In some examples additional structures (e.g., channels, other types of protrusions, another structure, or some combination thereof) are used to direct the flow of medication through the one or more output ports to further facilitate the flow of medication through the output region 319 and out of the medication bladder 310. In some examples, a plurality of output ports 306 of the one or more output ports may be used to direct the medication to different locations. As an example, a plurality of output ports 306 of the one or more output ports 306 may direct the medication to different injection points (e.g., locations on a patient's body configured for receipt of medication, such as by insertion of a needle). In some examples, at least one of the plurality of output ports 306 may be configured to output fluid or medication from the medication bladder to a cannula or other fluid channel, such as a cannula 1164 described with reference to FIG. 1. For example, an output port 306 may be configured to couple to a cannula or other fluid channel that may pass through or be engaged with a pump configured to cause a flow of medication or fluid from the medication bladder 310 towards an injection point on the patient. The coupling of the cannula and output port 306 may be a press fit or other connection type. In other examples, a cannula may be directly coupled to an output region 319 of the rigid portion 304.

Additionally, or alternatively, in examples utilizing a plurality of medication bladders 310 or a medication bladder 310 and at least one secondary fluid storage location, a plurality of output ports 306 of the one or more output ports 306 may be used to connect to additional medication bladders or fluid storage locations (e.g., connect an output port 306 of one medication bladder 310 to the input port 300 of another.) In further examples, the one or more output ports may be used to move medication between one or more medication bladders 310 or other fluid storage locations. For example, medication or other substance may be moved between the one or more medication bladders of a medication delivery system in order to deliver the medication to the patient as determined by a physician and/or by the medication delivery system. Advantageously, moving fluid between fluid storage locations, such as a medication bladder 310, may facilitate mixing or combining of medications or other substances prior to delivery to a patient.

As discussed, the rigid portion 304 may include an input region 322. The input region 322 may include an input or inlet port 300. The input region 322 may be located at least in part at a central region 311 of the rigid portion. In some examples, the input region 322 may be offset from the center. However, other configurations are also possible. For example, the input region 322 may be located at an edge 516 of the rigid portion 304. Additionally, or alternatively, the input region 322 could be located on a flexible portion 308 of the medication bladder 310. The input region 322 may additionally, or alternatively, protrude upward from a bulk or planar portion of the rigid portion 304, such as shown in FIG. 3.

In some examples, the input region 322 may include one or more input ports 300. The input port 300 may serve as an inlet into the medication bladder 310. An inlet seal 302 may be coupled to the input port 300. The input port 300 may be configured to receive the inlet seal 302. The inlet seal 302 might be configured to allow a fill device, such as a needle, to pass through it to fill the medication bladder 310 with medication. The inlet seal 302 may be able to self-close after the fill device is removed. The material of the inlet seal 302 may be a soft material, such as silicone. Which may comprise scalable opening and/or be configured to receive the inlet seal 302, as described above with regards to the input port 300. In some examples, the one or more input ports are used together to increase the rate of the flow of medication through the input region 322 and into the medication bladder 310. In some examples, additional structures (e.g., channels, other types of protrusions, another structure, a needle or needles, another fill device, or some combination thereof) may be used to help direct the flow of medication through the one or more input ports to further facilitate the flow of medication through the input region 322 and into the medication bladder 310.

FIG. 4 illustrates an example implementation of a rigid portion of an efficiently emptying medication bladder. With reference to FIG. 4, the interior surface 405 of the rigid portion 304 may be configured to help facilitate efficient emptying of the medication bladder 310. For example, the interior surface 405 may be configured to include one or more features to reduce unwanted retention of fluid within the medication bladder 310 during emptying. For example, the interior surface 405 may include an anti-aggregation coating so as to reduce aggregation of fluid including chemical substances (e.g., medications, additives, and the like) stored in the medication bladder 310. The coating may partially and/or substantially reduce aggregation of fluid including chemical substances included in the medication bladder 310. In some examples, the coating may be hydrophobic. In some examples, they hydrophobic nature of the coating may prevent aggregation of fluid by rejecting the fluid from holding on to the surface of the coating.

As illustrated in in FIG. 4, the rigid portion 304 may have a plurality of channels 400 configured to facilitate the flow of fluid out of the medication bladder 310. The plurality of channels 400 may extend throughout the rigid portion 304. The plurality of channels 400 may extend outward from a central region of the rigid portion 304 towards an edge 410 of the rigid portion 304. Additionally, or alternatively, the plurality of channels 400 may extend outward from the output region 319 towards the edge 410 of the rigid portion 304. Additionally, or alternatively, the plurality of channels 400 may extend outwards from the output port 306. In some examples, the plurality of channels 400 may extend outwards from the output region towards the edge of the rigid portion 304. The plurality of channels 400 may extend outward towards an edge 316 of the at least one rigid potion 304 radially from the central region 311 of the at least one rigid portion 304. However, other configurations and orientations are also possible.

The channels 400 may approximately follow the contour or curvature of the rigid portion 304 on the interior surface 405. As discussed, rigid portion 304 may include one or more guiding structures, such as curved or inclined surfaces. For example, the channels may be slightly curved towards a central depression 402, relatively flat in a central region 404, and more curved in a wall 318. As another example, the channels may be slightly curved towards a central depression 402, inclined in a central region 404 towards the wall 318, and more curved in a wall 318. The curved and/or inclined surfaces in the rigid portion 304 may facilitate the flow of fluid including chemical substances from the medication bladder. For example, the curvature of the curved portion (such as a wall 318) may be specified to mitigate the risk of fluid including chemical substances aggregating close to the edge 410 or the central depression 402. The curvature may apply a pressure to the one or more fluid including chemical substances in the medication bladder 310. The amount of pressure applied may depend on an amount of fluid including chemical substances in the bladder 310.

As referenced herein, the rigid portion 304 may be configured to couple to at least one flexible portion 308. In some examples, the at least one flexible portion 308 may be configured to change shape at least partially from a first shape or orientation into a second shape or orientation and/or any number of shapes or orientations between the first shape or orientation and the second shape or orientation. In some examples, the first shape may be of similar size and shape to a shape of the rigid portion 304. The second shape may be of similar size and shape to a general size and shape of the internal surface 405 of the rigid portion 304. However, the first shape and/or the second shape may not match or mirror the size and shape of the shape and/or surface(s) of the rigid portion. For example, the first and/or second shape may be smaller or larger than the size and shape of the rigid portion and/or include greater or fewer crevices, channels, or other components. In some implementations, the first shape may be approximately flat or planar and not configured to match the shape of the interior surface of the rigid portion 308.

In some implementations, the first shape of the flexible portion 308 may be oriented to mirror the orientation of the rigid portion 304 around a transverse plane or a point within a transverse plane formed by an edge 316, 317 at which the at least one rigid portion 304 and at least one flexible portion 308 meet or couple. The second shape may be configured to form an inverse orientation to the first shape and/or a parallel orientation to the at least one internal surface 405 of the rigid portion 304 when under negative pressure such that the second shape is configured to form an approximately parallel shape to the interior surface 405 of the rigid portion 304. As such, the medication bladder 310 may collapse so as to reduce an interior volume of the medication bladder 310, facilitating emptying of the medication bladder 310 when desired. As noted above, different amounts of pressure (such as negative pressure), may be configured to reduce the interior volume of the medication bladder 310 different amounts and/or expel different amounts of fluid held in the interior volume of the medication bladder 310.

The rigid portion 304 may be coupled to the flexible portion 308. The rigid portion 304 and the flexible portion 308 may be coupled in such a way that they form at least a partially sealed enclosure. In some examples, the rigid portion 304 and the flexible portion 308 may be coupled at an edge 316 or other area by a process that may include a weld or overmolding. In some examples, the joining process used to couple rigid portion 304 and flexible portion may comprise a laser weld. Additionally, or alternatively, the joining process used may comprise an ultrasonic weld. Additionally, or alternatively, the joining process used may comprise an RF weld. Additionally, or alternatively, the joining process may comprise overmolding. However, other methods of coupling the rigid portion 304 and the flexible portion 308 are also possible. In some examples, an edge 316 may be created during the weld or other coupling or joining process. In other examples, the edge 316 may be created prior to and independently of a coupling or joining process.

The dimensions as illustrated in FIG. 5A and FIG. 5B make it possible for the medication bladder 310 to fit into a minimally invasive device designed to comfortably sit on a person's body (e.g., on a person's abdomen, arm, etc.), such as the disease management system 1101 and/or the disease management system 1103, described in FIG. 1 and FIG. 2. Additionally, the dimensions as illustrated in FIG. 5A and FIG. 5B make it possible for the medication bladder 310 to include a volume of fluid including chemical substances sufficient to last for at least one or more days. For example, the medication bladder 310 may be configured to last two days, four days, 6 days, etc. The medication bladder 310 may even be configured to include an amount of fluid lasting for a week or more.

The size of the medication bladder 310 may be limited by the system into which its incorporated. For example, disease management system 1101 of FIG. 1 may be included inside of an enclosure. To ensure that other components of the disease management system 1101 also fit inside of the enclosure, the size of the medication bladder 310 may be limited to a specified range of dimensions. As another example, disease management system 1103 of FIG. 2 may be included inside of an enclosure. To ensure that other components of the disease management system 1101 also fit inside of the enclosure, the size of the medication bladder 310 may be limited to a specified range of dimensions. As a further example, a standard insulin delivery system may be inside of an enclosure. This may ensure that other components of the standard insulin delivery system fit inside the enclosure, the size of the medication bladder 310 may be limited to a specified range of dimensions.

In some examples, the volume of the medication bladder may be approximately 3 mL, such as 2.7 mL. However, other volumes are also possible. For example, the volume may be greater than 2.7 mL. Alternatively, the volume may be smaller than 2.7 mL. Changes in volume of the medication bladder 310 may change the duration of usage between refilling the medication bladder 310. For example, a larger volume may increase the duration of usage between refills of the medication bladder 310.

As illustrated by FIG. 5A, the width, W2, of the rigid portion 304 could be a width of W2. W2 could be a range of 18-30 mm. In one example, W2 could be approximately 20 mm. For example, W2 could be 20.15 mm. However, other dimensions are also possible.

As illustrated by FIG. 5A, the rigid portion 304 could have a region of width of W1. W1 could be a range of 18-30 mm. In one example, W1 could be approximately 18 mm, such as 18.18 mm. However, other dimensions are also possible.

As illustrated by FIG. 5A, the length of the rigid portion 304 could be a length of L2. L2 could be a range of 20-40 mm. In one example, L2 could be approximately 30 mm, such as 30.15 mm. However, other dimensions are also possible.

As illustrated by FIG. 5A, the length of the rigid portion 304 could have a region of length L1. L1 could be a range of 20-40 mm. In one example, L2 could be approximately 28 mm, such as 28.18 mm. However, other dimensions are also possible.

In some examples, the rigid portion 304 may have an edge 516. The edge 516 may correspond to edge 410 of FIG. 4. The edge 516 may have a dimension of L3. In some examples, L3 could be a range of 0-5 mm. In one example, L2 could be approximately 1 mm, such as 0.98 mm. In some examples, L3 may be equal to the difference between L2 and L1. However other dimensions are also possible.

Additionally, or alternatively, the edge 516 may have a dimension of W3. In some examples, W3 could be a range of 0-5 mm. In one example, L2 could be approximately 1 mm, such as 0.98 mm. In some examples, W3 may be equal to the difference between W2 and W1. However other dimensions are also possible. In some examples, L3 may be equal to W3. However, divergence in these measurements is also possible.

As illustrated by FIG. 5B, the edge 516 may have a height of H2. H2 may be a range of 5-15 mm. For example, H2 may be approximately 7 mm, such as 6.65 mm. However, other dimensions are also possible.

As illustrated by FIG. 5B, the rigid portion 304 may have a region with a height of H1. H1 may be a range of 0-10 mm. For example, H1 may be approximately 5 mm. However, other dimensions are also possible.

As illustrated by FIG. 5B, the edge 516 may have a height of H3. H3 may be a range of 0-5 mm. For example, H3 may be approximately 1 mm, such as 0.6 mm. In some examples, H3 may be equal to the difference between H2 and H1. However, other dimensions are also possible.

### Example Implementation of an Embodiment of a Medication Bladder

In some examples, the medication bladder 310 may be removable and/or replaceable from a system, such as a disease management system (e.g., disease management system 1101, disease management system 1103, etc.), a standard insulin pump or the like. After removal, a fill device may be inserted through the plug to fill the medication bladder 310 with one or more fluid including chemical substances. The fluid including chemical substances may include insulin and one or more additives to improved stability or delivery of the insulin. The additives may be included to control the release of insulin once injected into the patient. Additionally, or alternatively, the fluid including chemical substances may include glucagon and one or more additives to improved stability or delivery of the insulin. The additives may be included to control the release of glucagon once injected into the patient.

Additionally, or alternatively, the medication bladder 310 may be refilled without removing the medication bladder 310 from a system or while the medication bladder 310 is incorporated into a system, such as a disease management system (e.g., disease management system 1101, disease management system 1103, etc.), a standard insulin pump or the like. For example, the system may be included within an enclosure, such as a housing of a disease management system. Once the enclosure is opened the medication bladder 310 may be exposed. Specifically, the input port 300 with inlet seal 302 may be exposed. A user, such as a patient or caregiver, may then insert a fill device, such as a needle, to refill the medication bladder 310 with one or more fluid including chemical substances.

Additionally, or alternatively, the medication bladder 310 may receive fluid including chemical substances from other medication bladders 310. In some examples, utilizing one or more medication bladders 310, a plurality of input ports of the one or more input ports 300 may be used to connect to additional medication bladders 310 (e.g., connect an output port 306 of one medication port to the input port of another.) In further examples, the one or more input ports 300 may be used to receive fluid from one or medication bladders to facilitate the movement of fluid between one or more medication bladders. For example, the fluid may be moved between the one or more medication bladders of a medication delivery system in order to deliver the fluid to the patient as determined by a physician and/or by the medication delivery system.

With respect to the release of fluid from the medication bladder 310, in some examples, external pressure may be applied to a medication bladder 310, such as the medication bladder 310, to cause an amount of fluid to leave the medication bladder through an output port, such as the output port 306. In further examples, the external pressure may be applied by a pump, as discussed with respect to FIGS. 1-2 above. Types of pumps that may be used include, but are not limited to, piston pumps, peristaltic pumps, vacuum pumps, other kinds of pumps, or some combination thereof. In one example, the pump may be a vacuum pump. Additionally, or alternatively, a pressure applicator (e.g., pressure applicator 1178 of FIG. 2 may be used to apply external pressure to the medication bladder 310). In some examples, the pressure applicator may be in communication with one or more pumps. In other examples, the pressure applicator may be in communication with another type of force applicator, such as a motor. The pressure applicator may also be a motor.

In some examples, the flexible portion 308 may generate pressure against the rigid portion 304. The pressure may be generated with respect to the fluid inside the medication bladder 310. For example, if the medication bladder 310 is full, then the flexible portion 308 may apply a pressure resulting from the enclosure created by the rigid portion 304 and the flexible portion 308. For example, the fluid may cause a tension in the flexible portion 308. To release this tension, flexible portion 308 may apply a pressure on the fluid. In response to the pressure from the flexible portion 308, the fluid may flow through the output port 306 to a pump. Once at the pump, an external system, such as a disease management system or a standard insulin delivery system may be able to control delivery of the fluid to a patient in accordance with specified dosages, as described above with respect to FIGS. 1-2.

In some examples, at least part of the flexible portion 308 may move in response to the external applied pressure towards the rigid portion 304. The movement of the flexible portion 308 may apply a pressure to the fluid by compressing at least part of the flexible portion against the medication. The pressure generated by the flexible portion 308 may facilitate the flow of fluid through the output port 306. In further examples, the pressure generated by the flexible portion 308 may be generated through the compression of at least part of the flexible portion 308 against the rigid portion 304. The pressure generated by the flexible portion 308 may also be generated through the compression of at least part of the flexible portion 308 against additional structures (e.g., channels, other types of protrusions, curved surfaces (e.g., wall 318, curved portion 320, another structure, or some combination thereof) in the rigid portion 304.

Additionally, or alternatively, the shape of flexible portion 308 may depend on the fluid inside of medication bladder 310. For example, the flexible portion 308 may change shape as fluid is introduced or released from the medication bladder 310. How the flexible portion 308 changes shape may depend on the internal pressure caused by the presence or absence of fluid in the medication bladder. The shape of the flexible structure may additionally, or alternatively, be influenced by external pressures, such as those described above with respect to FIGS. 1-3.

In some examples, the plurality of channels 400 may further facilitate the flow of fluid by directing the fluid towards the output region 319. In some examples, internal pressures imposed by the flexible portion 308 and the one or more structures of rigid portion 304 may cause an amount of fluid including chemical substances to be directed to output port 306. The amount may be specified by a controller (e.g., controller 1138 of FIG. 1). In some examples, the pressure applied by the internal pressures may be sufficient to cause the release of fluid from the medication bladder 310 until the flexible portion 308 is flush with the bulk rigid portion 308. External pressure may be applied (e.g., by a pump, or pressure applicator) to bring the flexible portion flush with the plurality of channels to expel at least some of the fluid remaining in the medication bladder 310.

Additionally, or alternatively, the amount of fluid directed by internal pressure may be sufficient to bring the chemical substance in communication with a pump (e.g., pump 1130 of FIG. 1, pump 1186 of FIG. 2, etc.). Once in communication with a pump, the pump may apply external pressure to cause a specified amount of fluid to be delivered to a patient, to one or more other medication bladders, and the like. In some examples, a pressure applicator as described with reference to FIGS. 2-3 may additionally, or alternatively, be employed to apply external pressure to the medication bladder 310 to cause a specified amount of fluid to be delivered to a patient, to one or more other medication bladders, and the like.

In response to the external applied pressure, the flexible portion 308 may generate pressure against the rigid portion 304. In some examples, the pressure generated by the flexible portion 308 may also be generated through the compression of at least part of the flexible portion 308 against the plurality of channels 400. For example, the plurality of channels 400 may further facilitate the flow of fluid after the flexible portion 308 is flush with a bulk of the rigid portion 304 until the flexible portion 308 is also flush with the plurality of channels.

### Example Medication Delivery Flow

FIG. 6 illustrates an example routine 600, which may be performed by a disease management system. The routine 600 may be used to deliver fluid to a patient.

The routine 600 begins in block 602. The routine 600 may begin in response to an event such as the receipt by the disease management system of instructions from a user. In some examples, instructions may be entered into a user device (e.g., smart phone, smart watch, etc.) Additionally, or alternatively, instructions may be received from one or more controllers within the disease management system, as described in more detail above. When the routine begins at block 602, a medication bladder, such as the medication bladder 310, may be in a first fill state. The medication bladder in the first fill state may hold a first amount of fluid including chemical substances (e.g., medication, additives, or the like).

When the routine 600 is initiated, a set of executable program instructions stored on one or more non-transitory computer-readable media (e.g., hard drive, flash memory, removable media, etc.) may be loaded into memory (e.g., random access memory or RAM) of a computing system, such as the disease management system shown in FIG. 1, and executed by one or more processors. In some examples, the routine 600 or portions thereof may be implemented on multiple processors, serially or in parallel.

At block 604, the disease management system can receive instructions to deliver a particular bolus of fluid to the patient. In some examples, the particular bolus could be calculated by the controller, as described in more detail above. In further examples, the particular bolus may be calculated based in part on data from sensors applied to the patient. Historical sensor data may also be used in calculating the particular bolus. Additionally, or alternatively, this historical data may be stored in physical storage devices, such as solid-state memory chips and/or magnetic disks, into a different state. Additionally, or alternatively, this historical data may be stored on one or more remote storage devices. In some examples, the controller can verify whether the medication bladder holds an amount of fluid including chemical substances that is greater than the particular bolus. In further examples, the controller can verify whether the medication bladder in the first fill state holds a first amount of fluid that is greater than the particular bolus. Additionally, or alternatively, the controller may also verify whether the medication bladder requires a refill. For example, the controller may check the amount of medication against a threshold. The threshold may be predefined and/or set by the user. This threshold may be an approximate percentage of a maximum volume of a medication bladder, such as the medication bladder 310. For example, the threshold may be approximately 5% of the maximum volume of the medication bladder. In other examples, the threshold may be approximately 0% of the maximum volume of the medication bladder.

At block 606, the disease management system can determine a pressure to apply, using one or more pumps, to one or more medication bladders to release the particular bolus of fluid. In some examples, the pressure could be calculated by the controller. In further examples, the pressure may be calculated based in part on data from sensors applied to the patient. Historical sensor data may also be used in calculating the pressure. Additionally, or alternatively, this historical data may be stored in physical storage devices, such as solid state memory chips and/or magnetic disks, into a different state. Additionally, or alternatively, this historical data may be stored on one or more remote storage devices.

At block 608, the disease management system may send instructions to the one or more pumps to apply the pressure, as described with respect to FIGS. 1-2. Additionally, or alternatively, the disease management system may send instructions to one or more pressure applicators, as described with respect to FIG. 2. In some examples, instructions may be sent from one or more controllers within the disease management system, as described in more detail above. With reference to the illustrative embodiment of FIG. 3, in response to this external pressure the medication bladder 310 is configured to cause an amount of fluid, based on the particular bolus, to leave an outlet of the medication bladder 310, such as the output port 306.

In some examples, as described above, the medication bladder 310 may be configured such that at least part of the flexible portion 308 moves towards the rigid portion 304 in response to the pressure. The flexible portion 308 may apply a pressure against the fluid and/or against the rigid portion 304, which corresponds with the pressure applied by the pump.

After the amount of fluid leaves the medication bladder 310, the medication bladder may be in a second fill state. The second fill state may comprise a second amount of fluid. The second amount of fluid may be less than the first amount of fluid by approximately the amount of the particular bolus.

At block 610, the disease management system may verify whether the particular bolus has been delivered. For example, in some examples, the controller may access sensor data related to the one or more medication bladder (e.g., weight) to determine whether the particular bolus has been delivered. In some examples, this may comprise the medication bladder in the first fill state to the medication bladder in the second fill state. For example, the second amount of fluid may be compared to the first amount of fluid to determine whether the particular bolus has been delivered. In other examples, the controller may access sensor data relating to the patient to determine whether the fluid has taken effect as expected. As discussed, the fluid may include medication such as insulin, and the sensor data may indicate whether the insulin has been delivered. For example, the sensor data may include blood glucose level which the controller may compare to a prior blood glucose level to determine whether the insulin has had the desired effect. In further examples, the controller may use information on whether the fluid has taken effect as expected to determine whether to send instructions to provide an additional bolus of fluid to the patient. The amount of the particular bolus may be calculated by the controller, as described with respect to FIG. 1. In some examples, the additional bolus may be requested regardless of whether the delivery of the particular bolus has been verified. In some examples, verification of whether the particular bolus has been delivered, may not occur.

At block 612, the disease management system may verify whether any of the one or more medication bladders require refilling. As discussed above, in some examples, the controller may check the amount of medication against a threshold. For example, the second amount of medication of the medication in the second fill state may be checked against the threshold to determine whether a refill is required. The threshold may be preset or set by the user. This threshold may be an approximate percentage of a maximum volume of a medication bladder, such as the medication bladder 310. For example, the threshold may be approximately 5% of the maximum volume of the medication bladder. In other examples, the threshold may be approximately 0% of the maximum volume of the medication bladder. Additionally, or alternatively, the controller may access sensor data related to the one or more medication bladders (e.g., weight) to determine whether the one or more medication bladders require refill. In some examples, verification of whether the one or more medication bladders require refill, may not occur.

At block 614, the disease management system may request a refill of any of the one or more medication bladders. For example, in some examples, after determining whether any of the one or more medication bladders requires a refill, the disease management system may request a refill of any of the one or more medication bladders that were determined to require refill. Additionally, or alternatively, the disease management system can request a refill when any of the one or more medication bladders is determined to hold a lesser amount than a particular amount of fluid specified by the system and/or by a user. Additionally, or alternatively, the disease management system can request a refill of any of the one or more medication bladders after a set period of time has passed. The time period may be related to an expiration date of the fluid. In some examples, the period of time may be set by the controller of the disease management system. Additionally, or alternatively, the period of time may be set by a user (e.g., by entry into a user device, such as a smart phone or smart watch). However, it is also possible that the disease management system may not request refills.

Example Process for Release of Medication from a Medication Bladder

FIG. 7 illustrates an example process for release of medication from a medication bladder. The medication bladder may be similar to the medication bladder shown in FIGS. 3-5, as described in more detail above. The process begins at block 702. The process may begin in response to an event, such as configuration of the medication bladder for use in a disease management system, as described in more detail above.

At block 704, the medication bladder may receive an application of pressure from a medication delivery pump (e.g., pump 1130 of FIG. 1, pump 1186 of FIG. 2, etc.). The medication delivery pump may be a vacuum pump. Prior to application of pressure by the medication delivery pump, internal pressure provided by the surfaces and structures of the medication bladder, as described above with respect to FIGS. 3-5, may be applied to bring the medication into communication with the medication delivery pump. Additionally, or alternatively, the medication delivery pump may apply a small pressure to bring fluid including chemical substances (e.g., medications, additives, and the like) in communication with the medication delivery pump. Once in communication with the fluid, the medication delivery pump may then apply pressure to deliver a specified bolus. The applied pressure may be in accordance with instructions from a controller, such as controller 1138 of FIG. 1. The controller may provide instructions to achieve delivery a specified bolus to a specified destination, such as a patient or another medication bladder.

At block 706, the pressure applied by the medication delivery pump may be converted into a pressure applied by a flexible portion of the fluid to a rigid portion of a medication bladder. The flexible portion may comprise a soft film, as described in more detail above. With reference to FIGS. 3-5, in some examples, the applied pressure may cause the flexible portion 308 to begin to collapse against the rigid portion 304.

At block 708, the amount of fluid in the medication bladder may be at a level that requires compression of the flexible portion against a plurality of channels in the rigid portion to continue release of fluid from the medication bladder. With continued reference to FIGS. 3-5, If the flexible portion is flush with the bulk of the rigid portion 304, the applied pressure may need to cause the flexible portion 308 to compress against the channels 400 of FIG. 4.

At block 708, the flexible portion may compress against the plurality of channels in the rigid portion, as described in more detail above, to continue release of s. With continued reference to FIGS. 3-5, the compression may cause the fluid remaining in the channel to flow towards the outlet port 306. The flow of fluid including chemical substances from the medication bladder may be further be facilitated by anti-aggregation coatings on the interior surfaces of the flexible portion 308 and/or the rigid portion 304.

### Terminology

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning, which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a practitioner; however, they can also include any third-party instruction of those actions, either expressly or by implication.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

1-63. (canceled)

64. A disease management device comprising:
a pump configured to deliver fluid to an injection site of a patient;
one or more medication bladders configured to store the fluid prior to delivery to the injection site of the patient, each of the one or more medication bladders comprising:
a rigid portion coupled to a flexible portion to form an at least partially sealed enclosure, the rigid portion comprising:
an outlet port configured to allow an outward flow of the fluid from the at least partially sealed enclosure towards the pump,
wherein the flexible portion comprises a flexible material configured to change shape so as to facilitate movement of the fluid within the at least partially sealed enclosure towards the outlet port of the rigid portion.

65. The disease management device of claim 64,
wherein the rigid portion further comprises a first interior surface, and
wherein the flexible portion further comprises a second interior surface,
and wherein the first interior surface and second interior surface further comprise an anti-aggregation coating, wherein the anti-aggregation coating facilitates the movement of fluid within the at least partially sealed enclosure towards the outlet port of the rigid portion.

66. The disease management device of claim 64, the rigid portion further comprising:
an inlet port configured to facilitate access to the partially sealed enclosure for filling the medication bladder with the fluid.

67-68. (canceled)

69. The disease management device of claim 64, wherein the disease management device further comprises a pressure applicator configured to apply external pressure to at least one of the one or more medication bladders, wherein the applied external pressure is based on a force received by the pressure applicator from the pump.

70-73. (canceled)

74. The disease management device of claim 64, the rigid portion further comprising:
an interior surface comprising:
a plurality of channels configured to extend from at least one edge of the rigid portion towards the outlet port.

75. The disease management device of claim 74, wherein the rigid portion further comprises one or more protrusions, wherein each channel of the plurality of channels forms a depression within a protrusion of the one or more protrusions, and wherein the depression is configured to facilitate movement of fluid within the at least partially sealed enclosure towards the outlet port.

76. The disease management device of claim 74, wherein the rigid portion further comprises one or more guiding surfaces, including at least one of a curved surfaces or an inclined surface,
wherein the one or more guiding surfaces apply contours to the rigid portion to facilitate the movement of fluid within the partially sealed enclosure towards the outlet port of the rigid portion, and
wherein the plurality of channels follow the contours of the rigid portion.

77. The disease management device of claim 74, wherein the flexible material of the flexible portion is configured to change shape at least by compressing against the plurality of channels of rigid portion, wherein compression of the flexible material against the plurality of channels of the rigid portion facilitates the movement of fluid with the at least partially sealed enclosure towards the outlet port of the rigid portion.

78. (canceled)

79. The disease management device of claim 64, wherein the rigid portion further comprises:
one or more walls;
one or more edges;
a central region including the outlet port;
a top surface; and
one or more protruding structures,
wherein the one or more protruding structures extend vertically with respect to a major plane of the top surface,
wherein the one or more protruding structures extend from the at least of the one or more walls or one or more edges to the central region.

80. The disease management device of claim 79, wherein the one or more protruding structures vary in height with respect to a major plane of the top surface.

81. A medication bladder comprising:
a rigid portion coupled to a flexible portion to form an at least partially sealed enclosure, the rigid portion comprising:
an outlet port configured to allow an outward flow of fluid from the at least partially sealed enclosure; and
the flexible portion, wherein the flexible portion comprises a flexible material configured to change shape so as to facilitate movement of the fluid within the at least partially sealed enclosure towards the outlet port of the rigid portion.

82. The medication bladder of claim 81:
wherein the rigid portion further comprises a first interior surface,
wherein the flexible portion further comprises a second interior surface, and
wherein the first interior surface and second interior surface further comprise an anti-aggregation coating, wherein the anti-aggregation coating facilitates the movement of fluid within the at least partially sealed enclosure towards the outlet port of the rigid portion.

83. The medication bladder of claim 81, the rigid portion further comprising:
an inlet port configured to facilitate access to the partially sealed enclosure for filling the medication bladder with the fluid.

84. The medication bladder of claim 81, the rigid portion further comprising:
an interior surface comprising:
a plurality of channels configured to extend from at least one edge of the rigid portion towards the outlet port.

85. The medication bladder of claim 84, wherein the rigid portion further comprises one or more protrusions, wherein each channel of the plurality of channels forms a depression within a protrusion of the one or more protrusions, and wherein the depression is configured to facilitate movement of fluid within the at least partially sealed enclosure towards the outlet port.

86. The medication bladder of claim 84:
wherein the rigid portion further comprises one or more guiding surfaces, including at least one of a curved surfaces or an inclined surface,
wherein the one or more guiding surfaces apply contours to the rigid portion to facilitate the movement of fluid within the partially sealed enclosure towards the outlet port of the rigid portion, and
wherein the plurality of channels follow the contours of the rigid portion.

87. The medication bladder of claim 84, wherein the flexible material of the flexible portion is configured to change shape at least by compressing against the plurality of channels of rigid portion, wherein compression of the flexible material against the plurality of channels of the rigid portion facilitates the movement of fluid with the at least partially sealed enclosure towards the outlet port of the rigid portion.

88. The medication bladder of claim 81, wherein the rigid portion further comprises:
one or more walls;
one or more edges;
a central region including the outlet port;
a top surface; and
one or more protruding structures,
wherein the one or more protruding structures extend vertically with respect to a major plane of the top surface,
wherein the one or more protruding structures extend from the at least of the one or more walls or one or more edges to the central region.

89. The medication bladder of claim 88, wherein the one or more protruding structures vary in height with respect to a major plane of the top surface.

90. The medication bladder of claim 88:
wherein the rigid portion further comprises an interior surface,
wherein the interior surface includes a plurality of channels; and
wherein a location each channel on the interior surface corresponds to a location for a protruding structure on the top surface.
